(19) United States
(12) Patent Application Publication
(10) Pub. No.: US 2023/0263435 A1
(43) Pub. Date: Aug. 24, 2023

(54) ANALYTE SENSOR SYSTEM AND A METHOD FOR ITS PRODUCING

(71) Applicant: Roche Diabetes Care, Inc., Indianapolis, IN (US)

(72) Inventors: Peter Kettenmann, Mannheim (DE); Kirill Sliozberg, Mannheim (DE)

(73) Assignee: Roche Diabetes Care, Inc., Indianapolis, IN (US)

(21) Appl. No.: 18/307,071

(22) Filed: Apr. 26, 2023

Related U.S. Application Data
(63) Continuation of application No. PCT/EP2021/079456, filed on Oct. 25, 2021.

(30) Foreign Application Priority Data
Oct. 26, 2020 (EP) 20203820.4

(51) Int. Cl. A61B 5/145; A61B 5/1473

(52) U.S. Cl. CPC A61B 5/14546; A61B 5/1473; A61B 2562/12

(57) ABSTRACT

An analyte sensor system (110) and a method (160) for producing an analyte sensor system (110) are disclosed. Herein, the analyte sensor system (110) comprises:
an analyte sensor having
   a first contact pad, and
   a second contact pad;
a circuit carrier having
   a first contact area, and
   a second contact area, wherein the second contact area comprises at least two individual electrically conductive surfaces; and
a connecting element electrically connecting the second contact pad of the analyte sensor with each of the at least two individual electrically conductive surfaces of the second contact area of the circuit carrier.

The analyte sensor system (110) as proposed herein allows a reliable and persistent electrical contact between the analyte sensor (112) and the circuit carrier (114) which minimizes a risk of short circuits.

### FIELD OF THE INVENTION

The present invention relates to an analyte sensor system and a method for producing an analyte sensor system. The analyte sensor system may, primarily, be used for a long-term monitoring of an analyte concentration in a body fluid, in particular of a blood glucose level or of a concentration of one or more other analytes in the body fluid. The invention may be applied both in the field of home care as well as in the filed of professional care, such as in hospitals. However, other applications are feasible.

### RELATED ART

Monitoring body functions, in particular monitoring one or more concentrations of certain analytes, plays an important role in the prevention and treatment of various diseases. Without restricting further possible applications, the invention is described in the following with reference to glucose monitoring in an interstitial fluid. However, the invention can also be applied to other types of analytes. Blood glucose monitoring may, specifically, be performed by using electrochemical analyte sensors besides optical measurements. Examples of electrochemical analyte sensors for measuring glucose, specifically in blood or other body fluids, are known from U.S. Pat. Nos. 5,413,690 A, 5,762,770 A, 5,798,031 A, 6,129,823 A or US 2005/0013731 A1.

In the recent past, continuous measuring of glucose in the interstitial tissue, also referred to as “continuous glucose monitoring” or abbreviated to “CGM”, has been established as important method for managing, monitoring, and controlling a diabetes state. Herein, an active sensor region is applied directly to a measurement site which is, generally, arranged in an interstitial tissue, and may, for example, convert glucose into an electrically charged entity by using an enzyme, in particular glucose oxidase (GOD) and/or glucose dehydrogenase (GDH). As a result, the detectable charge may be related to the glucose concentration and can, thus, be used as a measurement variable. Examples thereof are described in U.S. Pat. No. 6,360,888 B1 or US 2008/0242962 A1.

Typically, current continuous monitoring systems are transcutaneous systems or subcutaneous systems. Accordingly, the analyte sensor or at least a measuring portion of the analyte sensor may be arranged under the skin of the user. However, an evaluation and control part of the system, which may also be referred to as a “patch”, may, generally, be located outside of the body of a user. Herein, the analyte sensor is generally applied by using an insertion instrument, which is, in an exemplary fashion, described in U.S. Pat. No. 6,360,888 B1. However, other types of insertion instruments are also known. Further, the evaluation and control part may, typically, be required which may be located outside the body tissue and which has to be in communication with the analyte sensor. Generally, communication is established by providing at least one electrical contact between the analyte sensor and the evaluation and control part, wherein the contact may be a permanent or a releasable electrical contact. Other techniques for providing electrical contacts, such as by appropriate spring contacts, are generally known and may also be applied.

In continuous glucose measuring systems, the concentration of the analyte glucose may be determined by employing an analyte sensor comprising an electrochemical cell having at least two individual electrodes. In particular, the analyte sensor may comprise two individual electrodes, i.e. a working electrode and a combined counter/reference electrode. As an alternative, the analyte sensor may comprise three individual electrodes, i.e. a working electrode, a counter electrode and a reference electrode. Alternatively, the three individual electrodes may be two working electrodes and a combined counter/reference electrode. As a further alternative, the analyte sensor may comprise at least four electrodes, i.e. at least two individual working electrodes and a combined counter/reference electrode or an individual counter electrode and an individual reference electrode. Herein, the at least one working electrode may have a reagent layer comprising an enzyme with a redox active enzyme co-factor adapted to support an oxidation of the analyte in the body fluid. During catalyzing the glucose oxidation, a reduction of the enzyme occurs, thus producing a reduced enzyme. Thereafter, at least one electrode signal is generated from which the analyte concentration can be determined. Further, the analyte sensor may comprise at least one contact for each electrode, especially, for establishing an electrical contact between each electrode and the evaluation and control part.

EP 2 621 339 B1 discloses systems and methods for processing, transmitting, and displaying data received from a continuous analyte sensor. Herein, the analyte sensor system comprises a sensor electronics module that includes power saving features, in particular a low power measurement circuit that can be switched between a measurement mode and a low power mode, in which charging circuitry continues to apply power to electrodes of a sensor during the low power mode. In addition, the sensor electronics module can be switched between a low power storage mode and a higher power operational mode via a switch. The switch can include a reed switch or an optical switch. A validation routine can be implemented to ensure an interrupt signal sent from the switch is valid. The continuous analyte sensor can be physically connected to a sensor electronics module.

### Problem to be Solved

It is therefore an objective of the present invention to provide an analyte sensor system and a method for producing an analyte sensor system, which at least partially avoid the shortcomings of known analyte sensors and related methods and which at least partially address the above-mentioned challenges.

In particular, it is desired that the analyte sensor system allows a reliable and persistent electrical contact between an analyte sensor and a circuitry designated for evaluation and control of the analyte sensor which minimizes a risk of short circuits.

### SUMMARY OF THE INVENTION

This problem is solved by an analyte sensor system and a method for producing an analyte sensor having system the features of the independent claims. Preferred embodiments of the invention, which may be implemented in an isolated way or in any arbitrary combination, are disclosed in the dependent claims and throughout the specification.

In a first aspect of the present invention, an analyte sensor system is disclosed, wherein the analyte sensor comprises:
an analyte sensor having
   a first contact pad, and
   a second contact pad;
a circuit carrier having
   a first contact area, and
   a second contact area, wherein the second contact area comprises at least two individual electrically conductive surfaces; and
a connecting element electrically connecting the second contact pad of the analyte sensor with each of the at least two individual electrically conductive surfaces of the second contact area of the circuit carrier,
wherein the second contact area (132) further comprises at least one electrically insulating surface (140) between the at least two individual electrically conductive surfaces (138, 138′) that jointly constitute the second contact area (132), and
wherein the analyte sensor (112) comprises an electrically insulating cover (134) which covers surfaces of the analyte sensor (112) apart from the first contact pad (124) and the second contact pad (128).

As generally used, the term “analyte sensor system” refers to an assembly of two or more components which are jointly configured for conducting at least one medical analysis. For this purpose, the analyte sensor system may be an arbitrary device configured for performing at least one diagnostic purpose and, for this purpose, comprises an analyte sensor for performing the at least one medical analysis. As described below in more detail, the analyte sensor system further comprises a circuit carrier and a connecting element.

Firstly, the analyte sensor system as disclosed herein comprises an analyte sensor. As further generally used, the term “analyte sensor” refers to an arbitrary device being configured to perform a detection of an analyte by acquiring at least one measurement signal. As particularly preferred, the analyte sensor may be a partially implantable analyte sensor which may, particularly, be adapted for performing the detection of the analyte in a body fluid of a user in a subcutaneous tissue, particularly in an interstitial fluid. As used herein, the term “partially implantable analyte sensor” refers to an arbitrary analyte sensor being adapted to be partly arranged within the body tissue of the patient or the user. For this purpose, the analyte sensor may comprise an insertable portion. Herein, the term “insertable portion” generally refers to a part or component of the analyte sensor which is configured to be insertable into an arbitrary body tissue. Other parts or components of the analyte sensor, in particular the contact pads, remain outside of the body tissue.

As generally used, both terms “user” and “patient” refer to a human being or an animal, independent from the fact that the human being or animal, respectively, may be in a healthy condition or may suffer from one or more diseases. As an example, the user or the patient may be a human being or an animal suffering from diabetes. However, additionally or alternatively, the invention may be applied to other types of users, patients or diseases.

As further used herein, the term “body fluid”, generally, refers to a fluid, in particular a liquid, which is typically present in a body or a body tissue of the user or the patient and/or may be produced by the body of the user or the patient. Preferably, the body fluid may be selected from the group consisting of blood and interstitial fluid. However, additionally or alternatively, one or more other types of body fluids may be used, such as saliva, tear fluid, urine or other body fluids. During the detection of the at least one analyte, the body fluid may be present within the body or body tissue. Thus, the analyte sensor may, specifically, be configured for detecting the at least one analyte within the body tissue.

As further used herein, the term “analyte” refers to an arbitrary element, component, or compound being present in the body fluid, wherein the presence and/or the concentration of the analyte may be of interest to the user, the patient, to medical staff, such as a medical doctor. In particular, the analyte may be or may comprise at least one arbitrary chemical substance or chemical compound which may participate in the metabolism of the user or the patient, such as at least one metabolite. As an example, the at least one analyte may be selected from the group consisting of glucose, cholesterol, triglycerides, lactate. Additionally or alternatively, however, other types of analytes may be used and/or any combination of analytes may be determined. The detection of the at least one analyte specifically may, in particular, be an analyte-specific detection. Without restricting further possible applications, the present invention is described herein with particular reference to a monitoring of glucose in an interstitial fluid.

In particular, the analyte sensor may be an electrochemical sensor. As used herein, the term “electrochemical sensor” refers to an analyte sensor which is adapted for a detection of an electrochemically detectable property of the analyte, such as an electrochemical detection reaction. Thus, for example, the electrochemical detection reaction may be detected by applying and comparing one or more electrode potentials. Specifically, the electrochemical sensor may be adapted to generate the at least one measurement signal which may, directly or indirectly, indicate a presence and/or an extent of the electrochemical detection reaction, such as at least one current signal and/or at least one voltage signal. The measurement may be a qualitative and/or a quantitative measurement. Still, other embodiments are feasible.

The electrochemical sensor as used herein is arranged in a fashion of an electrochemical cell and, thus, employs at least one pair of electrodes. As generally used, the term “electrode” refers to an entity of the test element which is adapted to contact the body fluid, either directly or via at least one semi-permeable membrane or layer. Each electrode may be embodied in a fashion that an electrochemical reaction may occur at at least one surface of the electrode. In particular, the electrodes may be embodied in a manner that oxidative processes and/or reductive processes may take place at selected surfaces of the electrodes. Generally, the term “oxidative process” refers to a first chemical or biochemical reaction during which an electron is released from a first substance, such an atom, an ion, or a molecule, which is oxidized thereby. A further chemical or biochemical reaction by which a further substance may accept the released electron is, generally, denominated by the term “reductive process”. Together, the first reaction and the further reaction may also be denominated as a “redox reaction”. As a result, an electrical current, which relates to moving electrical charges, may be generated hereby. Herein, a detailed course of the redox reaction may be influenced by an application of an electrical potential.

Further, each electrode comprises an electrically conductive material. As generally used, the term “electrically conductive material” refers to a substance which is designed for conducting an electrical current through the substance. For this purpose, a highly conductive material having a low electrical resistance is preferred, in particular to avoid a dissipation of electrical energy carried by the electrical current within the substance. Preferably, the electrically conductive material may be selected from a noble metal, especially gold; or from an electrically conductive carbon material; however, further kinds of conductive materials may also be feasible.

As further used herein, the term “determining” relates to a process of generating at least one representative result, in particular, by evaluating the at least one measurement signal as acquired by the analyte sensor. Herein, the term “evaluating” may refer to an application of methods for displaying the at least one measurement signal and deriving the at least one representative result therefrom. The at least one measurement signal may, specifically, be or comprise at least one electronic signal, such as at least one voltage signal and/or at least one current signal. The at least one signal may be or may comprise at least one analogue signal and/or may be or may comprise at least one digital signal. Especially in electrical systems, it may be required to apply a prespecified signal to a specific device in order to be able to record the desired measurement signal. By way of example, measuring a current signal may require the application of a voltage signal to the device, or vice-versa.

As further used herein, the term “monitoring” refers to a process of continuously acquiring data and deriving desired information therefrom without user interaction. For this purpose, a plurality of measurement signals are generated and evaluated, wherefrom the desired information is determined. Herein, the plurality of measurement signals may be recorded within fixed or variable time intervals or, alternatively or in addition, at an occurrence of at least one prespecified event. In particular, the analyte sensor as used herein may, especially, be configured for a continuous monitoring of one or more analytes, in particular of glucose, such as for managing, monitoring, and controlling a diabetes state.

In general, the analyte sensor may comprise a sensor body, in particular, a substrate. As used herein, the term “substrate” refers to an arbitrary element designed to carry one or more other elements disposed thereon or therein. Particularly preferred, the substrate may be a planar substrate. As generally used, the term “planar” refers to a body comprising extensions in two dimensions, typically denoted as “surface” of the planar body, which exceed the extension in a third dimension, usually denoted as “thickness” of the planar body, by a factor of 2, at least a factor of 5, at least a factor of 10, or even at least a factor of 20 or more. The substrate may, specifically, have an elongated shape, such as a strip shape or a bar shape; however, other kinds of shapes may also be feasible. As generally used, the term “elongated shape” indicates that each surface of the planar body has an extension in a direction along the elongation which exceeds an extension perpendicular hereto by at least a factor of 2, at least a factor of 5, at least a factor of 10, or even at least a factor of 20 or more. The substrate may comprise at least partially, preferably completely, at least one electrically insulating material, especially in order to avoid unwanted currents between electrically conducting elements as carried by the substrate. By way of example, the electrically insulating material may be selected from polyethylene terephthalate (PET) or polycarbonate (PC); however, other kinds of electrically insulating materials may also be feasible.

In particular, the substrate may be designed to carry the at least two contact pads as comprised by the analyte sensor, particularly denoted by the terms “first contact pad” and “second contact pad”. Herein, the terms “first” and “second” are considered as description without specifying an order and without excluding a possibility that other elements of that kind may be present. As used herein, the term “contact pad” refers to an element having at least one electrically conductive surface designated for transmitting measurement signals to and/or exchanging data with a circuitry designated for evaluation and control of the analyte sensor. In particular, the contact pad is configured to establish an electrical contact between a particular electrode of the analyte sensor and a corresponding contact area on the circuit carrier as described below in more detail. The circuit carrier may, fully or partially, carry the circuitry designated for evaluation and control of the analyte sensor, also denoted as “evaluation and control part”, and/or may comprise a further circuitry which is configured to transmit measurement signals and/or exchange data with the evaluation and control part.

The first contact pad and the second contact pad may be electrically conductive. As generally used, the term “electrically conductive” refers to a property of a substance of conducting an electrical current through the substance. Preferably, the first contact pad and/or the second contact pad may comprise a layer of an electrically conductive material. More preferred, the first contact pad and/or the second contact pad may comprise a layer of an electrically conductive material. As already defined above, the term “electrically conductive material” refers to a substance which is designed for conducting an electrical current through the substance. The electrically conductive material may, preferably, be selected from a noble metal, especially gold; or from an electrically conductive carbon material; however, further kinds of conductive materials may also be feasible.

In a particularly preferred embodiment of the present invention, the insertable portion of the analyte sensor may comprise the at least two electrodes which contact the body fluid, either directly or, in particular for a working electrode, via at least one semi-permeable membrane or layer. For a purpose of contacting the electrodes, each electrode can be arranged in a fashion that it may extend to the corresponding contact pad as comprised by the analyte sensor, preferably, outside of the body tissue. As an alternative, the analyte sensor may, in addition, comprise conductive traces which may be configured to provide the desired electrical contact between each electrode and the corresponding contact pad. Herein, the electrodes and, if applicable, the conductive traces may comprise the same electrically conductive material as the contact pads.

In a preferred arrangement, the first contact pad may be located on a first side of the analyte sensor, while the second contact pad may, concurrently, be located on a second side of the analyte sensor. As generally used, the term “side” refers to a surface of the sensor body. In a particularly preferred arrangement, the first contact pad and the second contact pad may be located on opposing sides of the analyte sensor. As generally used, the term “opposing sides” refers to the two planar surfaces of the flat substrate as described above or below in more detail. In this particularly preferred arrangement, the first contact pad and the second contact pad as comprised by the analyte sensor are not located within a common plane such that establishing the desired electrical contact between each contact pad and the corresponding contact area on the circuit carrier cannot be implemented by simply attaching both contact pads on to the contact area of the circuit carrier.

Further, the analyte sensor system as disclosed herein comprises a circuit carrier. As generally used, the term “circuit carrier” refers to a body provided for carrying at least one electronic, electrical, and/or optical element, in particular a plurality of such elements, wherein the carrier is designed to mechanically support and electrically connect the electronic, electrical, and/or optical elements. In a preferred embodiment, the circuit carrier may be a planar circuit carrier. As defined above, the term “planar” refers to a body which comprises extensions in two dimensions, typically denoted as “surface” of the planar body, which exceed the extension in a third dimension, usually denoted as “thickness” of the planar body, by a factor of 2, at least a factor of 5, at least a factor of 10, or even at least a factor of 20 or more. Alternatively, non-planar circuit carriers may also be applicable, in particular a flex printed circuit (FPC) or a mechatronic integrated device (MID).

In a particularly preferred embodiment, the circuit carrier may be or comprise a printed circuit board, usually abbreviated to “PCB”, which refers to an electrically non-conductive, planar substrate, also denoted as “board”, on which at least one sheet of an electrically conductive material, in particular a copper layer, is applied, specifically laminated, to the substrate, and which, in addition, comprises one or more electronic, electrical, and/or optical elements. Other terms which refer to this type of circuit carrier are printed circuit assembly, short “PCA”, printed circuit board assembly, short “PCB assembly” or “PCBA”, circuit card assembly, short “CCA”, or simply “card”. In the PCB, the electrically insulating substrate may comprise a glass epoxy, wherein a cotton paper impregnated with a phenolic resin, typically tan or brown, may also be used as substrate material. Depending on a number of sheets, the printed circuit board may be a single-sided PCB, a two-layer or double-sided PCB, or a multi-layer PCB, wherein different sheets may be connected with each other by using so-called “vias”. For the purposes of the present invention, an application of a single-sided PCB may be sufficient; however other kinds of printed circuit boards may also be applicable. A double-sided PCB may have metal on both sides, while a multi-layer PCB may be designed by sandwiching additional metal layers between further layers of electrically insulating material. Further, by using two double-sided PCBs, a four-layer PCB may be generated. In a multi-layer PCB, the layers can be laminated together in an alternating manner, such as in an order of metal, substrate, metal, substrate, metal, etc., wherein each metal layer may be individually etched and wherein any internal vias may be plated through before the multiple layers are laminated together. Further, the vias may be or comprise copper-plated holes which can, preferably, be designed as electrical tunnels through the electrically insulating substrate. For this purpose, through-hole components may also be used which may, usually, be mounted by wire leads passing through the substrate and soldered to tracks or traces on the other side.

Electrically conductive patterns or structures, such as tracks, traces, pads, vias for generating connections between adjacent sheets, or features such as solid conductive areas, may be introduced into the one or more sheets, preferably by removing a partition of the sheet, in particular by etching, silk screen printing, photoengraving, PCB milling, or laser resist ablation, at selected regions in the sheet, whereby the desired structures can be created. The etching can, preferably, be performed by using a photoresist material being coated onto the PCB which is, subsequently, exposed to light, whereby the desired pattern may be generated. Herein, the photoresist material may be adapted to protect the metal from dissolution into an etching solution. After etching, the PCB may, finally, be cleaned. By using this process, a particular PCB pattern can be mass-reproduced. However, other kinds of separation processes or connection processes may also be applicable. By way of example, a track introduced into the PCB may function as a wire being fixed at a selected position, wherein adjacent tracks can be insulated from each other, on one hand, by the substrate material and, on the other hand, by an electrically insulating fluid under conditions at which the PCB is used, specifically by air or a protective gas which may be present in a gap between the adjacent tracks. In addition, a surface of the PCB may have a coating, also denoted as a solder resist, which may be designed for protecting the metal, specifically the copper, within the at least one sheet from detrimental environmental effects, such as corrosion, thus, reducing a chance that undesired short circuits may be generated by a solder or by stray bare wires. In a multi-layer PCB, only outer metal layers may be coated in this manner since inner metal layers are protected by the adjacent substrate layers.

Further, the electronic, electrical, and/or optical elements or components may be placed onto the substrate, such as by soldering, welding, or depositing, or, additionally or as an alternative, be embedded into the circuit carrier, such as by placing them into seats designated in the substrate for this purpose and/or by deliberately removing a partition of the circuit carrier. Preferably, surface mount components, specifically transistors, diodes, IC chips, resistors and capacitors, may, thus, be attached to the PCB by using electrical conductive leads which adjoin the respective component to metal tracks, traces, or areas on the same side of the substrate. As an alternative, through-hole mounting may be used, in particular, for extended or voluminous components, such as electrolytic capacitors or connectors. As a further alternative, the components may be embedded within the substrate. In addition, the PCB may, further, comprise an area on the PCB, usually denoted by the term “silkscreen”, on which an identifying text, such as a legend identifying the components or test points, may be printed. However, other kinds of circuit carriers may also be applicable.

In particular, the circuit carrier comprises two contact areas, particularly denoted by the terms “first contact area” and “second contact area”. Again, the terms “first” and “second” are considered as description without specifying an order and without excluding a possibility that other elements of that kind may be present. As used herein, the term “contact area” refers to an element having at least one electrically conductive surface designated for receiving measurement signals from and/or exchanging data with the analyte sensor. The first contact area and the second contact area may be electrically conductive. Preferably, the first contact area and/or the second contact area may comprise a layer of an electrically conductive material. More preferred, the first contact area and/or the second contact area may consist of a layer of an electrically conductive material. For the terms “electrically conductive” and “electrically conductive material” reference can be made to the definitions as provided above. The electrically conductive material as used for the contact areas may, preferably, be identical with the electrically conductive material as used for the contact pads, especially in order to minimize contact resistances. The electrically conductive material may, preferably, be selected from a noble metal, especially gold; or from an electrically conductive carbon material; however, further kinds of conductive materials may also be feasible.

According to the present invention, the second contact area comprises at least two individual electrically conductive surfaces. In other words, the second contact area as comprised by the circuit carrier has two or more electrically conductive surfaces which are separated from each other. As used herein, the term “separated” refers to an arrangement in which each individual electrically conductive surface is located in a distance towards the at least one further individual electrically conductive surface. In particular, the distance between the at least two individual electrically conductive surfaces may be selected in a fashion to impede a flow of an electrical current between the at least two individual electrically conductive surfaces via the surface of the second contact area. For this purpose, at least one electrically insulating surface may be provided on the surface of the circuit carrier between the at least two individual electrically conductive surfaces which jointly constitute the second contact area of the circuit carrier. In particular, at least one layer of an electrically insulating material may, preferably, be arranged as the at least one electrically insulating surface on the surface of the circuit carrier between the at least two layers of an electrically conductive material which form the at least two individual electrically conductive surfaces jointly constituting the second contact area of the circuit carrier. In other words, the second contact area may be or comprise a split contact area. Herein, any suitable arrangement for the split contact area may be selected, in particular an axial symmetric layout or a concentric co-axial design, wherein, however, further kinds of arrangements may also be feasible.

In a particular embodiment, the at least two individual electrically conductive surfaces which jointly form the second contact area of the circuit carrier can be comprised by at least two individual electrically conducting elements which are electrically separated from each other in a fashion to impede the flow of an electrical current between the at least two individual electrically conducting elements. However, in a further preferred embodiment, apart from the at least one electrically insulating surface located between the at least two individual electrically conductive surfaces, the second contact area of the circuit carrier may be a coherent electrically conducting element. For this purpose, the at least two layers of the electrically conductive material may be electrically connected with respect to each other, however, below the at least one electrically insulating surface on the surface of the circuit carrier. In other words, the at least two individual electrically conductive surfaces of the second contact area may electrically be connected with each other outside, preferably only outside, the surface of the at least two individual electrically conductive surfaces. This further preferred embodiment can, advantageously, facilitate the transfer of measurement signals from and/or data between the second contact pad of the analyte sensor and the second contact area of the circuit carrier.

The first contact area may, preferably, be or comprise a single electrically conductive element, such as by having a single layer of electrically conductive material, which comprises a single and coherent electrically conductive surface. However, in a particular embodiment, it may also be feasible that the first contact area, similar to the second contact area, can comprise at least two individual electrically conductive surfaces, especially in fashion as described in connection with the second contact area.

Further, the analyte sensor system as disclosed herein comprises a connecting element. As generally used, the term “connecting element” refers to an arbitrary element which is designated for establishing an electrical contact between at least two individual elements which are fully or at least partially electrically conducting. In particular, the connecting element is designated for electrically connecting the second contact pad of the analyte sensor with each of the at least two individual electrically conductive surfaces of the second contact area of the circuit carrier. For this purpose, the single connecting element may, preferably, exhibit a lateral extension which may allow touching at least a portion, preferably the whole surface, of all individual electrically conductive surfaces that jointly form the second contact area and, more preferred, also at least one adjacent portion of the electrically insulating surface on the surface of the circuit carrier which adjoins the individual electrically conductive surfaces.

The connecting element may be selected from a connecting element which comprises at least one of an electrically conductive rubber; an electrically conductive foam; an elastomeric connector. However, further kinds of connecting element may also be feasible. As generally used, the terms “elastomeric connector” or “zebra connector” refer to a particular connecting element which comprises electrically conductive regions and electrically insulating regions in an alternating fashion, especially by using a rubber matrix or an elastomer matrix in order to generate anisotropic conductive properties.

In the particularly preferred arrangement as already indicated above, the first contact pad and the second contact pad as comprised by the analyte sensor may be located on opposing sides of the analyte sensor. In particular, the analyte sensor may be placed in a manner within the analyte sensor system with respect to the surface of the circuit carrier that the first contact pad faces the first contact area of the circuit carrier while the second contact pad faces away from the second contact area of the circuit carrier. In other words, the sensor body of the analyte sensor may, typically, be placed in a fashion that a first side of the sensor body which carries the first contact pad may face the surface of the circuit carrier which carries both the first contact area and the second contact area, while a second side of the sensor body which carries the second contact pad may face away from the same surface of the circuit carrier. According to this arrangement, the first contact pad and the second contact pad as comprised by the analyte sensor in this arrangement are not located within a common plane.

On one hand, the desired electrical contact between the first contact pad of the analyte sensor and the first contact area of the circuit carrier which directly face each other may be generated in a straightforward manner by directly attaching the first contact pad onto the first contact area of the circuit carrier. In other words, the analyte sensor may be located in a fashion that the first contact pad may directly connect the circuit carrier. In a preferred embodiment, the electrically conducting surfaces of both the first contact pad and the first contact area may be pressed against each other, in particular, in order to maintain a reliable and persistent electrical contact between the first contact pad and the first contact area. For a purpose of generating a compressive force between the two facing surfaces of the first contact pad and the first contact area, an additional electrically non-conductive elastic element can be applied. In addition, it may, however, be feasible, to introduce here a further connecting element, which may be similar to the connecting element between the second contact pad and the second contact area as described above or below in more detail.

On the other hand, the connecting element is used for electrically connecting the second contact pad of the analyte sensor with each of the at least two individual electrically conductive surfaces of the second contact area of the circuit carrier. As a result of this particular arrangement, an undesired electrical contact and, thus, a possible short circuit between the first contact pad and the second contact area of the circuit carrier can be avoided. As schematically illustrated in the examples below, the first contact pad may exhibit a frayed edge that may otherwise produce an unwanted short circuit. As generally used, the term “frayed edge” refers to a particular shape of a boundary of an object having an irregular form which may, typically, be generated during producing of the object. In particular, the frayed edges can be generated by cutting of a substrate which comprises more than one analyte sensor into individual analyte sensors. Moreover, the frayed edges can be generated by using an electrically conductive foamed material having a rough surface which can abrade the insulating layer.

In particular, the electrically conducting material as comprised by the first contact pad may exhibit an irregular shape at the boundary of the first contact pad which may, unavoidably, be generated during producing of the analyte sensor, especially, during applying of the electrically conducting material onto the substrate of the analyte sensor. Although the first contact pad of the analyte sensor may have frayed edges capable of contacting the second contact area, the particular arrangement according to the present invention ensures that the frayed edges of the first contact pad can only contact an electrically insulating portion of the second contact area outside the at least two individual electrically conductive surfaces of the second contact area of the circuit carrier. As a result, no short circuit can, advantageously, occur between the analyte sensor and the circuit carrier.

In order to, additionally, support this particular advantage, the connecting element may, preferably, exhibit a form which may allow covering at least the electrically conductive surfaces and, more preferably, at least one adjacent portion of the electrically insulating surface on the surface of the circuit carrier. For further details, reference can be made to the examples as described below.

In a further particular embodiment, the analyte sensor may comprise an electrically insulating cover, which may cover surfaces of the analyte sensor apart from the first contact pad and the second contact pad. In particular, the electrically insulating cover may be an electrically insulating varnish, such as a photoresist or a solder resist; however, a further kind of electrically insulating cover may also be feasible. This particular embodiment further supports protecting the analyte sensor but still allows generating the desired electrical contacts between each contact pad on the analyte sensor and each corresponding contact area on the circuit carrier.

In a further aspect of the present invention, a method for producing an analyte sensor system, in particular for producing the analyte sensor system as described herein, is disclosed. The method comprises the following steps of:
a) providing a circuit carrier having
   a first contact area, and
   a second contact area, wherein the second contact area comprises at least two individual electrically conductive surfaces;
b) arranging an analyte sensor having
   a first contact pad, and
   a second contact pad;
   and
c) applying a connecting element in a manner that the connecting element electrically connects the second contact pad of the analyte sensor with each of the at least two individual electrically conductive surfaces of the second contact area of the circuit carrier.

Herein, the indicated steps may, preferably, be performed in the given order, thereby commencing with step a), continuing with step b), and finishing with step c). Further, additional steps, whether described herein or not, may be performed, too.

In a preferred procedure, the circuit carrier which is provided during step a) may comprise at least one casing that may have a plurality of structural elements which may be designated for supporting the arranging of the analyte sensor according to step b) and the applying of the connecting element according to step c). Herein, the structural elements may, in particular, be selected from at least one of a notch, a recess, an indentation, a slot, an edge, or a protrusion; however, further kinds of structural elements may also be feasible.

In a further preferred procedure, step b) may further comprise arranging the analyte sensor in a manner that the first contact pad may directly connect the first contact area of the circuit carrier. As described above and below in more detail, the electrically conducting surfaces of both the first contact pad and the first contact area may, in a preferred embodiment, be pressed against each other, for which purpose an additional electrically non-conductive elastic element can be applied.

In a further preferred procedure, the analyte sensor as provided during step b) may comprise an electrically insulating cover, such as an electrically insulating varnish, which may be provided in a fashion that it may cover surfaces of the analyte sensor apart from the first contact pad and the second contact pad, thereby maintaining the desired electrical contacts between each contact pad on the analyte sensor and each corresponding contact area on the circuit carrier.

For further details with regard to the method, reference can be made to the description of the analyte sensor system above or below.

The analyte sensor system and the related method according to the present invention exhibit a particular advantage with respect to the prior art in that the analyte sensor system as proposed herein minimizes a risk of a short circuit by allowing a reliable and persistent electrical contact between the analyte sensor and the circuit carrier. Herein, the circuit carrier may, fully or partially, carry the evaluation and control part and/or may comprise a further circuitry which is configured to transmit measurement signals and/or exchange data with the evaluation and control part, such that the analyte sensor system as proposed herein, particularly, qualifies for a use in a continuous glucose monitoring system which demands a reliable and persistent operation over a long period of time.

In contrast hereto, EP 2 621 339 B1 discloses a connector pad of the sensor electronics module which is configured to contact at least one corresponding contact of a mounting unit and which can be split into two individual, electrically insulated connectors. Herein, the contact of the mounting unit can be in form of a conductive, flexible “puck”, designed to make contact with a corresponding “split” connector of the sensor electronics module when the sensor electronics module is attached to the mounting unit. Once in contact, the split connector and the conductive puck result in a short circuit. This can cause sensor electronics module to switch on after an impedance measurement or switch on a battery voltage to wake up the sensor electronics module. In contrast hereto, the analyte sensor system according to the present invention avoids generating a short circuit.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. Herein, in most cases, when referring to the respective feature or element, the expressions “at least one” or “one or more” will not be repeated, non-withstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarizing, the following embodiments are potential embodiments of the present invention. However, other embodiments may also be feasible.

An analyte sensor system, comprising
an analyte sensor having
   a first contact pad, and
   a second contact pad;
a circuit carrier having
   a first contact area, and
   a second contact area, wherein the second contact area comprises at least two individual electrically conductive surfaces; and
a connecting element electrically connecting the second contact pad of the analyte sensor with each of the at least two individual electrically conductive surfaces of the second contact area of the circuit carrier.

Embodiment 1. The analyte sensor system according to the preceding Embodiment, wherein the second contact area further comprises at least one electrically insulating surface between the at least two individual electrically conductive surfaces that jointly constitute the second contact area.

Embodiment 2. The analyte sensor system according to any one of the preceding Embodiments, wherein the at least two individual electrically conductive surfaces of the second contact area are electrically connected with each other outside the surface of the second contact area.

Embodiment 3. The analyte sensor system according to any one of the preceding Embodiments, wherein the at least two individual electrically conductive surfaces of the second contact area are electrically connected with each other only outside the surface of the second contact area.

Embodiment 4. The analyte sensor system according to any one of the preceding Embodiments, wherein the first contact pad is located on a first side of the analyte sensor.

Embodiment 5. The analyte sensor system according to any one of the preceding Embodiments, wherein the second contact pad is located on a second side of the analyte sensor.

Embodiment 6. The analyte sensor system according to any one of the preceding Embodiments, wherein the first contact pad and the second contact pad are located on opposing sides of the analyte sensor.

Embodiment 7. The analyte sensor system according to any one of the preceding Embodiments, wherein the analyte sensor is located in a manner that the first contact pad faces the first contact area of the circuit carrier.

Embodiment 8. The analyte sensor system according to any one of the preceding Embodiments, wherein the analyte sensor is located in a manner that the second contact pad faces away from the second contact area of the circuit carrier.

Embodiment 9. The analyte sensor system according to any one of the preceding Embodiments, wherein the analyte sensor is located in a manner that the first contact pad directly connects the first contact area of the circuit carrier.

Embodiment 10. The analyte sensor system according to any one of the preceding Embodiments, wherein the analyte sensor comprises an electrically insulating cover which covers surfaces of the analyte sensor apart from the first contact pad and the second contact pad.

Embodiment 11. The analyte sensor system according to the preceding Embodiment, wherein the electrically insulating cover is or comprises an electrically insulating varnish.

Embodiment 12. The analyte sensor system according to any one of the preceding Embodiments, wherein at least one of the first contact pad, the second contact pad, the first contact area, and the second contact area comprises a layer of an electrically conductive material.

Embodiment 13. The analyte sensor system according to the preceding Embodiment, wherein at least one of the first contact pad, the second contact pad, the first contact area, and the second contact area consists of a layer of an electrically conductive material.

Embodiment 14. The analyte sensor system according to any one of the two preceding Embodiments, wherein the conductive material is selected from at least one of gold and an electrically conductive carbon material.

Embodiment 15. The analyte sensor system according to any one of the preceding Embodiments, wherein the connecting element comprises at least one of
an electrically conductive rubber;
an electrically conductive foam;
an elastomeric connector.

Embodiment 16. The analyte sensor system according to any one of the preceding claims, wherein the circuit carrier is or comprises a printed circuit board (PCB).

Embodiment 17. The analyte sensor system according to any one of the preceding claims, wherein the analyte sensor is a partially implantable analyte sensor for continuously monitoring an analyte.

Embodiment 18. The analyte sensor system according to any one of the preceding Embodiments, wherein the analyte sensor is an analyte sensor for continuously monitoring an analyte.

Embodiment 19. The analyte sensor system according to any one of the preceding Embodiments, wherein the analyte sensor is an analyte sensor for a continuous measurement of the analyte in a subcutaneous tissue.

Embodiment 20. The analyte sensor system according to any one of the preceding Embodiments, wherein the analyte sensor is an analyte sensor for a continuous measurement of the analyte in a body fluid.

Embodiment 21. The analyte sensor system according to any one of the preceding Embodiments, wherein the analyte sensor is an analyte sensor for a continuous measurement of the analyte in an interstitial fluid.

Embodiment 22. The analyte sensor system according to any one of the preceding Embodiments, wherein the analyte sensor is an analyte sensor for a continuous measurement of the analyte in blood.

Embodiment 23. The analyte sensor system according to any one of the preceding Embodiments, wherein the analyte sensor is configured to convert the analyte into an electrically charged entity by using an enzyme.

Embodiment 24. The analyte sensor system according to any one of the preceding Embodiments, wherein the analyte comprises glucose.

Embodiment 25. The analyte sensor system according to the preceding Embodiment, wherein the analyte sensor is configured to convert glucose into an electrically charged entity by using an enzyme,

Embodiment 26. The analyte sensor system according to the preceding Embodiment, wherein the enzyme is at least one of glucose oxidase or glucose dehydrogenase.

Embodiment 27. A method for producing an analyte sensor system, in particular the analyte sensor system according to any one of the preceding claims, the method comprising the steps of:
a) providing a circuit carrier having
   a first contact area, and
   a second contact area, wherein the second contact area comprises at least two individual electrically conductive surfaces;
b) arranging an analyte sensor having
   a first contact pad, and
   a second contact pad;
   and
c) applying a connecting element in a manner that the connecting element electrically connects the second contact pad of the analyte sensor with each of the at least two individual electrically conductive surfaces of the second contact area of the circuit carrier.

Embodiment 28. The method according to the preceding Embodiment, wherein step b) further comprises arranging the analyte sensor in a manner that the first contact pad directly connects the first contact area of the circuit carrier.

Embodiment 29. The method according to the preceding Embodiment, wherein step b) further comprises generating a compressive force between the first contact pad and the first contact area by applying an additional electrically non-conductive elastic element.

Embodiment 30. The method according to any one of the preceding Embodiments referring to the method, further comprising providing an electrically insulating cover for covering surfaces of the analyte sensor apart from the first contact pad and the second contact pad.

### SHORT DESCRIPTION OF THE FIGURES

Further details of the invention can be derived from the following disclosure of preferred embodiments. The features of the embodiments can be implemented in an isolated way or in any combination. The invention is not restricted to the embodiments. The embodiments are schematically depicted in the Figures. The Figures are not to scale. Identical reference numbers in the Figures refer to identical elements or functionally identical elements or elements corresponding to each other with regard to their functions.

In the Figures:

FIG. 1 schematically illustrates a top view of an analyte sensor system according to the present invention, the analyte sensor system comprising an elongate analyte sensor, a circuit carrier, and a connecting element;

FIG. 2 schematically illustrates a cross-sectional view of the analyte sensor system through the analyte sensor in a direction along an elongation of the elongate analyte sensor;

FIG. 3 schematically illustrates a cross-sectional view of the analyte sensor system through the connecting element in a direction perpendicular to the elongation of the elongate analyte sensor;

FIG. 4 schematically illustrates a section of the analyte sensor system as shown in FIG. 3 in an enlarged version in order to demonstrate a particular advantage of the analyte sensor system according to the present invention; and

FIG. 5 schematically illustrates a method for producing an analyte sensor system according to the present invention.

### DETAILED DESCRIPTION OF THE EMBODIMENTS

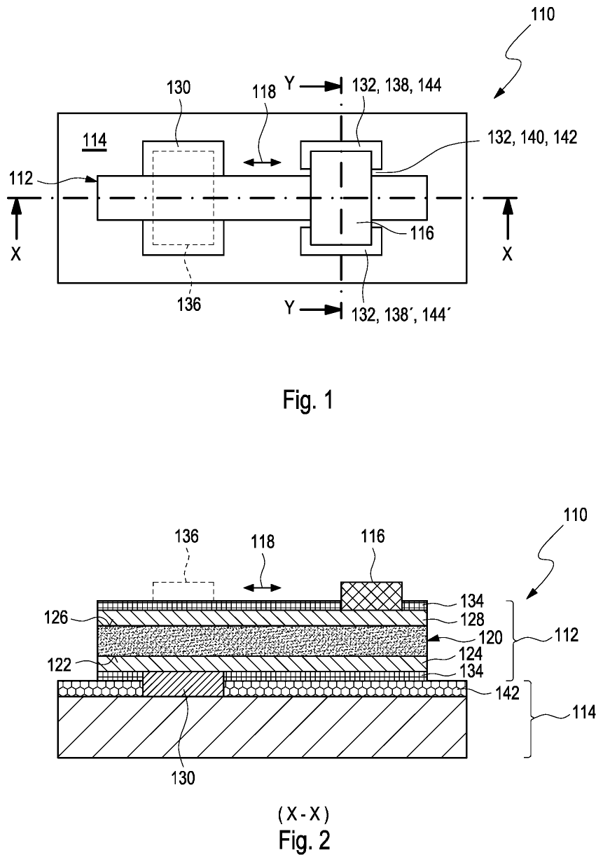

FIGS. 1 to 3 each schematically illustrates an analyte sensor system 110 according to the present invention, wherein the analyte sensor system 110 as proposed herein comprises an analyte sensor 112, a circuit carrier 114, and a connecting element 116. The analyte sensor 112 may be a partially implantable analyte sensor for continuously monitoring an analyte, in particular by performing a continuous measurement of the analyte in a subcutaneous tissue, preferably in a body fluid, especially in an interstitial fluid or in blood. For this purpose, the analyte sensor 112 may be configured to convert the analyte into an electrically charged entity by using an enzyme. Specifically, the analyte may comprises glucose, which may be converted into an electrically charged entity by using at least one of glucose oxidase (GOD) or glucose dehydrogenase (GHD) an the enzyme, However, the analyte sensor system 110 according to the present invention may also be applicable to other kinds of analytes as well as to other processes for monitoring an analyte.

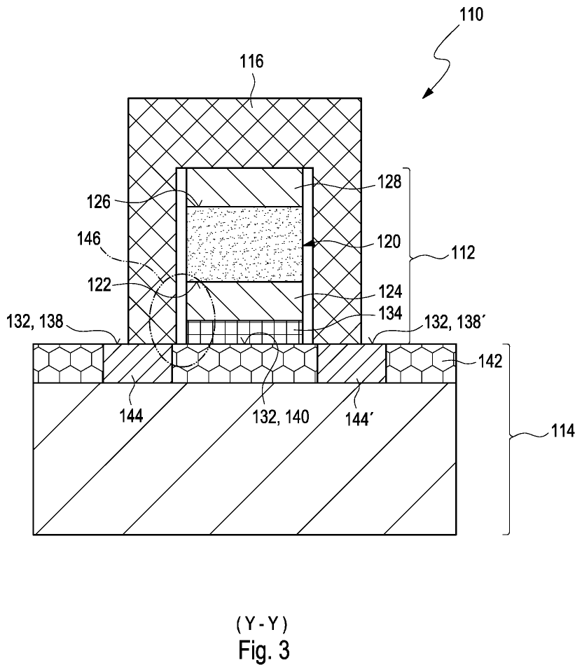

FIGS. 1 to 3 each illustrates a portion of the analyte sensor 112 which remains outside of the body tissue. In the particular example of FIGS. 1 to 3 this is a portion of an elongate analyte sensor; however, other forms of the analyte sensor 112 may also be feasible. While FIG. 1 illustrates a top view of the analyte sensor system 110, FIG. 2 shows a cross-sectional view of the analyte sensor system 110 along line X—X as indicated in FIG. 1 through the analyte sensor 112 in a direction along an elongation 118 of the elongate analyte sensor 112 and FIG. 3 depicts a cross-sectional view of the analyte sensor system 110 along line Y—Y as indicated in FIG. 1 through the connecting element 116 in a direction perpendicular to the elongation 118 of the elongate analyte sensor 112.

As further illustrated in FIGS. 2 and 3, the analyte sensor 112 comprises a substrate 120 having a first side 122 on which a first contact pad 124 is located and a second side 126 on which a second contact pad 128 is located. As already described above, each contact pad 124, 128 is configured to establish an electrical contact between a particular electrode of the analyte sensor 112 and a corresponding contact area on the circuit carrier 114. In the exemplary embodiment as used herein, an insertable portion of the analyte sensor 112 may comprise at least two electrodes (not depicted here) which are configured to contact the body fluid, either directly or, in particular for a working electrode, via at least one semi-permeable membrane or layer. For a purpose of contacting the electrodes, each electrode can be arranged in a fashion that it may extend to the corresponding contact pad 124, 128. As an alternative, the analyte sensor 112 may, in addition, comprise conductive traces (not depicted here) which may be configured to provide the desired electrical contact between each electrode and the corresponding contact pad 124, 128.

As schematically depicted, the first contact pad 124 and the second contact pad 128 are located on opposing sides 122, 126 of the substrate 120. In the particular example of FIGS. 1 to 3, the substrate 120 is a planar substrate; however, other forms may also be feasible. Further, the substrate 120 has an elongated shape, in particular a bar shape; however, other kinds of shapes may also be feasible. In particular, the substrate 120 may be an electrically insulating substrate which, preferably, comprises at least one electrically insulating material, especially to avoid unwanted currents between the contact pads 124, 128. By way of example, the electrically insulating material may be selected from polyethylene terephthalate (PET) or polycarbonate (PC); however, other kinds of electrically insulating materials may also be feasible.

As further illustrated in FIG. 2, the first contact pad 124 faces a first contact area 130 as comprised by the circuit carrier 114 while the second contact pad 128 faces away from a second contact area 132 of the circuit carrier 114. The circuit carrier 114 may, preferably, be or comprise a printed circuit board (PCB) as described above in more detail. However, other kinds of circuit carriers may also be feasible. Further, each of the first contact pad 124, the second contact pad 128, the first contact area 130, and the second contact area 132 comprises, preferably consists of, a layer of an electrically conductive material which may, specifically, be selected from gold and/or conductive carbon; however other kinds of conductive materials may also be feasible.

As depicted in FIGS. 1 to 3, the analyte sensor 112 may, additionally, comprise an electrically insulating cover 134, such as an electrically insulating varnish, such as a photoresist or a solder resist, which may cover surfaces of the analyte sensor 112 apart from the first contact pad 124 and the second contact pad 128, thereby maintaining the desired electrical contacts between each contact pad 124, 128 and each corresponding contact area 130, 132. However, the present invention also refers to analyte sensor systems 110 which do not comprise the electrically insulating cover 134 at all or not in the particular arrangement as depicted in FIGS. 1 to 3.

In the example as depicted in FIGS. 1 and 2, the first contact pad 124 directly connects the first contact area 130 of the circuit carrier 114. As indicated by dashed lines, a compressive force can be generated between the two facing surfaces of the first contact pad 124 and the first contact area 130 by applying an additional electrically non-conductive elastic element 136. However, the present invention also refers to analyte sensor systems 110 which do not comprise the electrically non-conductive elastic element 136.

As further schematically illustrated in FIGS. 1 and 3, the second contact area 132 as comprised by the circuit carrier 114 has at least two individual electrically conductive surfaces 138, 138′ according to the present invention. For this purpose, the second contact area further comprises, as shown in FIG. 3, an electrically insulating surface 140 between the two individual electrically conductive surfaces 138, 138″, which jointly constitute the second contact area. For this purpose, the electrically insulating surface 140 may be comprised by an electrically insulating layer 142 which is, as further depicted in FIG. 3, arranged between two layers 144, 144′ of an electrically conductive material which provide the two individual electrically conductive surfaces 138, 138′. As a result, the second contact area 132 may be considered as a split contact area, wherein any suitable arrangement for the split contact area may be selected, in particular an axial symmetric layout or a concentric co-axial design. However, further kinds of arrangements for the split contact area may also be feasible. In a particular embodiment (not illustrated here), the two individual electrically conductive surfaces 138, 138′ of the second contact area 132 may, still, be electrically connected with each other, however, only outside the surface of the second contact area 132.

As further shown in FIGS. 1 to 3, the analyte sensor system 110 comprises the connecting element 116. In accordance with the present invention, the connecting element 116 is designated for electrically connecting the second contact pad 128 of the analyte sensor 112 with each of the two individual electrically conductive surfaces 138, 138′ of the second contact area 132 as comprised by the circuit carrier 114. The connecting element 116 may assume a suitable form which can, as especially depicted in FIG. 3, provide a reliable and persistent electrical connection between the second contact pad 128 of the analyte sensor 112, which faces away from the second contact area 132, and both individual electrically conductive surfaces 138, 138′ of the second contact area 132. Further, the connecting element 116 may comprise an electrically conductive material as already described above, in particular, selected from an electrically conductive rubber, an electrically conductive foam, or an elastomeric connector, also denoted as “zebra connector”. However, other kinds of connecting elements may also be feasible.

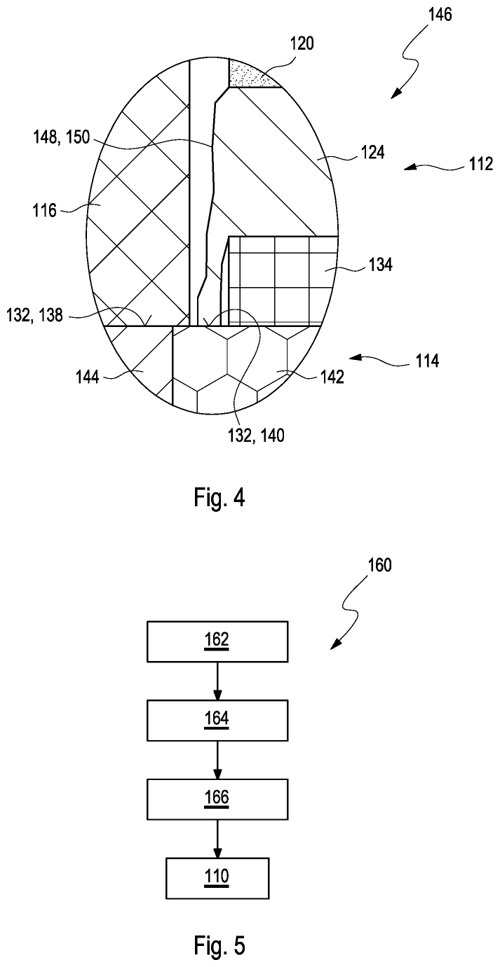

FIG. 4 schematically illustrates a section 146 of the analyte sensor system 110 as indicated in FIG. 3 in an enlarged version in order to demonstrate a particular advantage of the analyte sensor system 110 as result of the particular arrangement in accordance with the present invention. As depicted there, the edge 148 of the first contact pad 124 as comprised by the analyte sensor 112 may be or comprise a frayed edge. As a result of the frayed edge, electrically conductive material 150 may reach the surface of the second contact pad 132 comprised by the circuit carrier 114. Due to the particular arrangement of the analyte sensor system 110 in accordance with the present invention, the electrically conductive material 150 as provided by the frayed edge 148 of the first contact pad 124 can only reach the electrically insulating surface 140 comprised by the electrically insulating layer 142 as further depicted in FIG. 4. The electrically conductive surface 138 as comprised by the layer 144 of the electrically conductive material is located in a region that cannot be reached by the electrically conductive material 150 of the frayed edge 148. Moreover, the form of the connecting element 116 can be chosen to cover at least the electrically conductive surfaces 138, 138′ and, preferably, at least one adjacent portion of the electrically insulating surface 140. As a result, unwanted short circuits between the analyte sensor 112 and the circuit carrier 114 can be avoided in a reliable and persistent manner. It is emphasized here that this particular advantage of the analyte sensor system 110 according to the present invention also occurs in embodiments which do not comprise the electrically insulating cover 134 at all or not in the particular arrangement as depicted in FIGS. 1 to 4.

FIG. 5 schematically illustrates a method 160 for producing the analyte sensor system 110 according to the present invention.

In a providing step 162 according to step a), the circuit carrier 114 having the first contact area 130 and the second contact area 132 is provided, wherein the second contact area 132 comprises two individual electrically conductive surfaces 138, 138′.

In an arranging step 164 according to step b), the analyte sensor 112 having the first contact pad 124 and the second contact pad 128 is arranged, preferably on top of the circuit carrier 114.

In an applying step 166 according to step c), the connecting element 116 is applied in a manner that the connecting element 116 electrically connects the second contact pad 132 of the analyte sensor 112 with each of the at least two individual electrically conductive surfaces 138, 138′ of the second contact area 132 of the circuit carrier 114.

In a preferred embodiment, the arranging step 164 may further comprise arranging the analyte sensor 112 in a manner that the first contact pad 124 directly connects the first contact area 130 of the circuit carrier 114.

In a further preferred embodiment, the arranging step 164 may, additionally, comprise generating a compressive force between the first contact pad 124 and the first contact area 130 by applying the additional electrically non-conductive elastic element 136.

In a particularly preferred embodiment as described above, in a covering step (not depicted here) the electrically insulating cover 134 may be provided in a fashion that it may cover surfaces of the analyte sensor 112 apart from the first contact pad 124 and the second contact pad 128, whereby the electrical contacts between each contact pad 124, 128 and each corresponding contact area 130, 132 may not be impaired.

### LIST OF REFERENCE NUMBERS

110 analyte sensor system
112 analyte sensor
114 circuit carrier
116 connecting element
118 elongation
120 substrate
122 first side
124 first contact pad
126 second side
128 second contact pad
130 first contact area
132 second contact area
134 electrically insulating cover
136 electrically non-conductive elastic element
138, 138′ individual electrically conductive surface
140 electrically insulating surface
142 electrically insulating layer
144, 144′ layer of electrically conductive material
146 section
148 edge
150 electrically conductive material
160 method for producing an analyte sensor system
162 providing step
164 arranging step
166 applying step

1. An analyte sensor system, comprising
an analyte sensor having
a first contact pad, and
a second contact pad;
a circuit carrier having
a first contact area, and
a second contact area, wherein the second contact area comprises at least two individual electrically conductive surfaces; and
a connecting element electrically connecting the second contact pad of the analyte sensor with each of the at least two individual electrically conductive surfaces of the second contact area of the circuit carrier, wherein the second contact area further comprises at least one electrically insulating surface between the at least two individual electrically conductive surfaces that jointly constitute the second contact area, and wherein the analyte sensor comprises an electrically insulating cover which covers surfaces of the analyte sensor apart from the first contact pad and the second contact pad.

2. The analyte sensor system according to claim 1, wherein the at least two individual electrically conductive surfaces of the second contact area are electrically connected with each other outside the surface of the second contact area.

3. The analyte sensor system according to claim 1, wherein the first contact pad is located on a first side of the analyte sensor, and wherein the second contact pad is located on a second side of the analyte sensor.

4. The analyte sensor system according to claim 1, wherein the first contact pad and the second contact pad are located on opposing sides of the analyte sensor.

5. The analyte sensor system according to claim 1, wherein the first contact pad faces the first contact area of the circuit carrier, and wherein the second contact pad faces away from the second contact area of the circuit carrier.

6. The analyte sensor system according to claim 1, wherein the first contact pad directly connects the first contact area of the circuit carrier.

7. The analyte sensor system according to claim 1, wherein at least one of the first contact pad, the second contact pad, the first contact area, and the second contact area comprises a layer of an electrically conductive material.

8. The analyte sensor system according to two claim 6, wherein the conductive material is selected from at least one of gold or an electrically conductive carbon material.

9. The analyte sensor system according to claim 1, wherein the connecting element comprises at least one of
an electrically conductive rubber;
an electrically conductive foam;
an elastomeric connector.

10. The analyte sensor system according to claim 1, wherein the circuit carrier is or comprises a printed circuit board.

11. The analyte sensor system according to claim 1, wherein the analyte sensor is a partially implantable analyte sensor for continuously monitoring an analyte.

12. A method for producing an analyte sensor system, in particular the analyte sensor system according to claim 1, the method comprising the steps of:
a) providing a circuit carrier having
a first contact area, and
a second contact area, wherein the second contact area comprises at least two individual electrically conductive surfaces;
b) arranging an analyte sensor having
a first contact pad, and
a second contact pad;
and
c) applying a connecting element in a manner that the connecting element electrically connects the second contact pad of the analyte sensor with each of the at least two individual electrically conductive surfaces of the second contact area of the circuit carrier.

13. The method according to claim 12, wherein step b) further comprises arranging the analyte sensor in a manner that the first contact pad directly connects the first contact area of the circuit carrier.
